(19) United States
(12) Patent Application Publication
(10) Pub. No.: US 2011/0089370 A1
(43) Pub. Date: Apr. 21, 2011

(54) Lipophilic Preparations

(75) Inventors: Bernhard Gutsche, Hilden (DE); Ulrich Schörken, Dusseldorf (DE); Albrecht Weiss, Langenfeld (DE); Bernd Fabry, Korschenbroich (DE)

(73) Assignee: COGNIS IP MANAGEMENT GMBH, Dusseldorf (DE)

(21) Appl. No.: 12/672,705

(22) PCT Filed: Aug. 1, 2008
(86) PCT No.: PCT/EP2008/006338

§ 371 Date: Jan. 5, 2011

(30) Foreign Application Priority Data
Aug. 10, 2007 (DE) 10 2007 037 783.7

(51) Int. Cl. C12P 7/64; C09K 3/00

(52) U.S. Cl. 252/182.12; 435/134

(57) ABSTRACT

Disclosed are novel lipophilic preparations comprising (a) from 20 to 40% by weight of myristic acid or esters thereof, (b) from 20 to 40% by weight of palmitic acid or esters thereof, (c) from 0.1 to 5% by weight of aliphatic and/or cycloaliphatic hydrocarbons and (d) less than 20% by weight of carboxylic acids or esters thereof having 12 and fewer carbons in the acyl moiety and (e) less than 20% by weight of carboxylic acids or esters thereof having 16 and more carbons in the acyl moiety, with the proviso that all percentages add up to 100% by weight.

### FIELD OF THE INVENTION

The invention is in the field of oleochemical basic substances and relates to novel lipid preparations which are obtained on the basis of specific microorganisms.

### STATE OF THE ART

Fatty acids and esters thereof are important raw materials for a large number of industries and are used as preproducts especially for detergent surfactants, lubricants and cosmetic ingredients. With vegetable oils and animal fats, nature produces a lasting, ecologically and economically valuable source of raw materials which, having the multitude of available fatty acid spectra, meet a large number of industrial requirements. And yet the prior art is a long way from providing a satisfactory solution for all existing problems.

A fundamental shortcoming with the industrial production of fatty acids and derivatives thereof through cleavage and/or transesterification of fats and oils is in particular that nature only provides in available amounts those raw materials which comprise a surplus of long-chain saturated and unsaturated fatty acids and also of short-chain species, but comprise only comparatively small amounts of myristic acid (C14) and palmitic acid (C16). However, it is precisely these fatty acids which have the optimum carbon chain length for cosmetic application and also for use in detergents.

Besides these framework conditions, which are prescribed by the market, there is a need to improve, in an economically and ecologically permanent manner, the processes specified at the outset for producing fatty acids and alkyl esters thereof as first products in the value-adding chain. Apart from the raw material costs, the expenditure for energy is nowadays the greatest contributor to the production costs. Thus, for example, even shortening the reaction time by a few minutes or reducing the temperature by a few degrees Celsius during the production of mass-produced products leads to considerable energy savings and thus cost savings. The technical problems during the production of fatty acids and esters thereof which have hitherto been awaiting a solution include the removal of glycerol following the cleavage and/or transesterification, which always necessitates a time-consuming phase separation under economically acceptable framework conditions.

The complex object of the present invention was therefore to provide lipophilic preparations which firstly meet market requirements, i.e. have a high fraction of C₁₄- and C₁₆-fatty acid(s) (derivatives), and secondly have the advantage of a simplified industrial production compared with the prior art.

### DESCRIPTION OF THE INVENTION

The invention provides lipophilic preparations comprising
(a) 20 to 40, preferably 25 to 30, % by weight of myristic acid or esters thereof,
(b) 20 to 40, preferably 25 to 30, % by weight of palmitic acid or esters thereof,
(c) 0.1 to 5, preferably 0.5 to 1, % by weight of aliphatic and/or cycloaliphatic hydrocarbons, and
(d) less than 20, preferably less than 15, % by weight of carboxylic acids or esters thereof having 12 and fewer carbons in the acyl radical and
   with the proviso that all of the percentages add up to 100% by weight. Moreover, the quantitative data relating to (a) and (b) are to be understood such that the total amount of these two components constitutes 40 to 80% by weight, where the individual species can also, if appropriate, fall below or exceed the limits stated above.

Surprisingly, it has been found that the preparations according to the invention satisfy the profile of requirements explained at the start in an excellent manner. The lipid fractions have firstly the high fraction of C₁₄- and C₁₆-fatty acid(s) (derivatives), whereas the longer-chain and shorter-chain species are only represented in small amounts. The content of hydrocarbons leads to the separating off of the glycerol following the cleavage of fat and/or transesterification being able to be shortened by 10%, which leads to a significant increase in plant capacity and also to a reduction in energy consumption. Further interesting products of value, such as, for example, sterols or vitamins, are likewise present in the lipid fractions and, following separation and purification, can improve the profitability. The most important finding on which this invention is based, however, is that exactly such preparations are directly produced by certain microorganisms, specifically algae. Moreover, it is particularly important that these microorganisms already have sufficiently high lipid contents for commercial exploitation as raw material source to be possible and for a strain selection or genetic modification to lead merely to an increase in the profitability of the process without forming a prerequisite therefor. Finally, the invention permits the use of waste materials (carbon dioxide from combustion plants, waste waters from starch processing) as nutrient media for the cell cultures, which makes recycling possible, in which harmful emissions from other processing plants reduced.

The preparations are furthermore characterized in that the components (a), (b), (d) and (e) are present independently of one another either as full or partial glycerides or as esters with aliphatic alcohols having 1 to 4 carbon atoms. The hydrocarbons which form the component (c) are primarily squalene or squalane.

### Production Processes

The invention further provides a process for producing the aforementioned lipophilic preparations, in which
(a) lipid-producing single- or multi-celled microorganisms are cultivated which
   (a1) have a lipid content—based on dry mass—of at least 10, preferably at least 25 and in particular 40 to 60, % by weight, where
   (a2) the lipid fraction
      (a21) 20 to 40, preferably 25 to 30, % by weight of myristic acid or esters thereof,
      (a22) 20 to 40, preferably 25 to 30, % by weight of palmitic acid or esters thereof,
      (a23) 0.1 to 5, preferably 0.5 to 1, % by weight of aliphatic and/or cycloaliphatic hydrocarbons, and
      (a24) less than 20, preferably less than 15, % by weight of carboxylic acids or esters thereof having 12 and fewer carbons in the acyl radical and
      (a25) less than 20, preferably less than 15, % by weight of carboxylic acids or esters thereof having 16 and more hydrocarbons in the acyl radical,
(b) the microorganisms are subjected to an extraction in which the lipid fraction is separated off from the biomass.

### Microorganisms

The microorganisms which serve within the context of the present invention as starting materials for the production of the C_{14/16}-fatty acid-rich lipid fractions are preferably so-called microalgae or μ-algae. These are eukaryotic, phototropic, predominantly aquatic microorganisms which, with the help of chlorophylls and light energy, produce organic substances from inorganic substances. They are divided into the following classes:
Crytophyceae
Dinophyceae
Prymnesiophyceae
Chrysophyceae
Bacillariophyceae
Dictyochophyceae
Euglenophyceae
Chlorophyceae

Typical examples of particularly suitable microorganisms, especially microalgae and specifically microalgae from the genus Chrysophycea, are:
Tetraselmis suecica,
Nannochloropsis,
Dinobryon divergens,
Mallomonas caudata,
Syncrypta globosa,
Synura urella,
Haptophycea isochrysis,
Chaeto ceros,
Paulova lutheri,
Isochrysis galbana,
Emiliana huxleyi and
Prymnesiophycea parvum,
Isochrysis
   which, even without optimization of the cultivation conditions or mutagenesis or genetic engineering methods, have myristic/palmitic acid contents of 30 to 70%. The aforementioned representatives are known from the prior art. Thus, their lipid composition is reported, for example, in von Mourente et al. [Hydrobiologia 203, 147 (1990)], Gamido et al. [J. Phycol. 36, 497 (2000)], Cobelas et al. [Grasas y Aceites 40, 118 (1989)] or Elias et al. [Aquacultural Eng. 29, 155 (2003)], but these do not go into ways as to how precisely the desired content of C_{14/16}-fatty acids can be increased or what significance the content of hydrocarbons could have with regard to the processing properties of the lipid fractions. Alternatively, diatoms, such as, for example, Skeletonema costatum or Phaeodactylum tricornutum, and also fungi, such as, for example, Pythium, are also suitable.

### Cultivation

The optimum cultivation of the microorganisms represents an important framework condition for the technical teaching of the present invention since only rapid algae growth and high lipid contents render the realization economically useful. Although it is directly possible to cultivate, for example, the specified microalgae under conventional conditions, as a rule the lipid amounts obtainable thereby, based on the total dry mass, turn out to be too low to make industrial exploitation attractive—at least without suitable methods for concentration.

A large number of different cultivation conditions and nutrient media both for small and industrial cultures of algae cells are known from the prior art. Within the context of the present invention, however, it has proven to be particularly advantageous to start from the following conditions. The temperature at which the algae cultures are cultivated is, for example, one of the particularly critical parameters since the species can originate from different biotopes—inland waters or open sea, warm or cold regions—and can therefore have very different preferences. Usually, however, constant heat-treatment of the cultures at 20 to 40° C. with an optimum of about 30° C. leads to particularly advantageous results. In this connection, it has been found that the higher temperature generally results in a higher fraction of saturated fatty acids.

In a further preferred embodiment of the present invention, the algae are cultivated mixotrophically, i.e. with the addition of additional nutrients for the cell growth. For this purpose, the so-called “Arnon medium” has proven particularly advantageous, especially if it is enriched with nitrates in amounts of about 10 to 60 mM and preferably about 40 mM. The growth rate can likewise be increased by adding salts of acetic acid, in particular sodium acetate, the amount added typically being in the range from 20 to 60 mM and preferably about 40 mM. It is of course possible to also use mixtures of nitrates and acetates. However, a further advantage also consists in the fact that the specified microalgae also permit the use of very cost-effective nutrient media, for example waste waters from the dairy or starch processing, which are available in large amounts virtually free of charge and consequently make the process according to the invention additionally attractive.

The irradiation of the algae is obviously likewise a critical parameter. In this connection, it is on the one hand to be ensured that the algae receive an adequate amount of light without, on the other hand, being shaded excessively. In the pilot plant, an amount of light of from 200 to 1500 μm⁻² s⁻¹ and preferably 700 to 1000 μm⁻² s⁻¹, has proven to be particularly advantageous. On the production scale, it would of course be preferable to manage with natural irradiation.

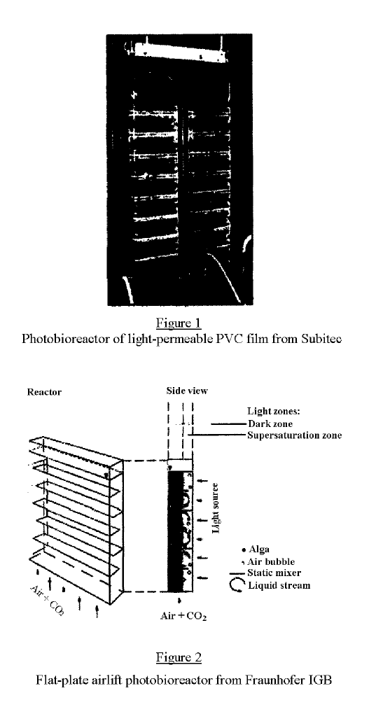

A particular problem with the cultivation of algae consists in the fact that, as the concentration of algae mass increases, the incident amount of light can only penetrate a few centimeters into the suspension, which leads to lower layers being virtually no longer irradiated. In order to prevent this, a further preferred embodiment of the present invention consists in cultivating the cells in a photobioreactor, preferably a tubular or flat-plate photobioreactor. These reactors have the particular advantage that, relative to their volume, they have a particularly large surface area, meaning that the cultures in each case only form thin layers of about 4 to 5 cm and are therefore optimally irradiated. The volume of such reactors can be between 100 and 50000 l, depending on the production amount, with both glass and plastics, such as, for example, polyacrylates, polycarbonates or PVC, being suitable as materials. Corresponding device are known from the prior art and are sold and/or supplied, for example, by the companies iq-Bradenburg (Biostat BPR 3500), Subitec or by Fraunhofer IGB; two corresponding reactors is shown by way of example in FIGS. 1 and 2.

During the growth phase, the cell suspensions are pumped through the reactor, the water being enriched with CO₂. This serves in particular the purpose of preventing clumping of the masses. A further advantage with regard to the selected microalgae is that these directly also permit the use of unpurified carbon dioxide, for example from combustion plants. This can reduce harmful emissions and, conversely, even emission certificates can be earned.

During the cultivation, the cell concentration in the photobioreactor should preferably be adjusted to 0.1 to 0.3*106 cells/ml at a concentration of 0.1 to 0.4 g of dried biomass/l, which can be achieved, for example, through the controlled addition of fresh nutrient medium. As has already been explained above, the amount of light should preferably be 200 to 1500 μm⁻² s⁻¹ and preferably 700 to 1000 μm⁻² s⁻¹, as is supplied, for example, by mercury vapor lamps. In the photobioreactor, moreover, the temperature should be kept at 20 to 35° C. As explained above, the temperature is, as it were, a switch for the degree of saturation of the fatty acid mixtures obtained in this way.

As explained at the start, although the aforementioned conditions are completely suitable for a conventional cultivation of the microalgae, it may be necessary to alter the conditions in order to stimulate the algae to increased production of lipids, i.e. to improve the yields and to make the process more profitable. This applies in particular where although the algae have already been optimized in respect of the fatty acid spectrum, the amount of lipid, when considered absolutely, is too low. Such a stimulation can take place through stress factors which trigger in the algae a reflex to increased formation of storage substances. Within the context of the process according to the invention, very different factors are suitable for this purpose:
increasing the light intensity
withdrawing nutrients
chemical and/or oxidative stress, and
changing the pH.

Surprisingly, it has been found that, besides the periodic introduction and cutting back of nutrients, in contrast to the customary expert opinion, chemical stress leads, in particular, to increased synthesis of storage lipids. Here, in particular the addition of peroxidic compounds, such as hydrogen peroxide, in amounts of from 10 to 50 mM has proven effective.

### Separation and Processing

The separation and processing of the lipid fraction from the remaining biomass is also of high importance. By means of efficient separation processes, it is possible to at least partly compensate for the disadvantage of low lipid concentrations—it of course being obvious that the combination of high lipid contents and optimized processing is preferred.

Within the context of the invention, the preferred method consists in sedimenting, filtering and/or centrifuging the suspensions in order to separate off the algae mass from the nutrient medium. For this, it has proven useful to add standard commercial flocculating agents to the suspensions. Usually, for this, following each cultivation cycle of 4 to 6 days, the biomass is let out of the photobioreactor and left to its own devices in a sedimentation vessel for several hours in order to facilitate separation. The ratio between the still moist biomass and the supernatant aqueous nutrient medium solution is here usually 30:70. The aqueous phase can be drawn off and returned to the cycle, while the residue is preferably dried in a centrifuge or in a vacuum filter and concentrated. The amount of dry mass here is 30 to 40%, based on the starting mass, depending on the chosen process.

Alternatively, the lipid fractions can also be removed from the algae by a “milking process”. For this, the algae suspensions are treated with an organic solvent and the lipid fractions are extracted. This offers the advantage that the algae can be returned again to the photoreactor and be reused for further lipid synthesis. A corresponding process is the subject of the international patent application WO 03/095397 A2/A3 (Cognis), the contents of which are hereby incorporated by reference.

### INDUSTRIAL APPLICABILITY

According to the teaching of the present invention, lipid fractions are obtained which have a high content of C14 and C16-fatty acids, whereas shorter- and longer-chain species are present in only small amounts. The lipid fractions also comprise amounts of hydrocarbons and additional products of value, such as, for example, sterols, squalenes and vitamins. Following the concentration, the lipid fractions can be freed from the residual water, for which purpose in particular spray-drying or freeze-drying present themselves. The powders obtainable in this way can be used immediately for a variety of intended uses. However, as a rule, it is desirable, instead of the lipids, to obtain the fatty acids or lower alkyl esters thereof.

### Fatty Acid Isolation

To isolate the fatty acids from the lipids, the concentrates can be subjected in a manner known per se either to an esterification or to a transesterification with lower alcohols.

One option consists in subjecting the suspensions to a thermal, chemical or preferably enzymatic hydrolysis, in which case the free fatty acids migrate directly from the cell membranes into the liquid phase, where they can be separated off from the biomass by sedimentation, centrifugation or filtration. The biomass can be combusted and in so doing then produces energy for the further process. Since it is a non-fossil fuel, there is the further advantage here that CO₂ certificates are not used up, but, conversely, are earned, which makes the process both ecologically and economically of interest. The fatty acid fraction can then be esterified with lower alcohols, preferably methanol, in a manner known per se, and then be fractionated and/or purified in order to provide the desired C-cuts.

In an alternative embodiment of the present invention, the suspensions can also be directly further processed by chemical means without prior mechanical separation and processing. For this, a reaction of the lipid fractions in the suspension with lower alcohols is one possible option, in which case the corresponding alkyl, preferably methyl, esters are obtained directly. This can take place, for example, under pressure in a twin-screw extruder, as is described for the extraction of oil seeds in the European Patent Application EP 0967264 A1 (Toulousaine de Recherche et Developpement AB). Within the context of the present invention, for this purpose, preferably the algae suspensions are placed in an extruder with the lower alcohol—preferably methanol—and a standard commercial transesterification catalyst and, in this way, a mechanical disruption of the cell membranes with release of the lipids is achieved. The mixture is then heated until a glycerol phase is formed. The partially transesterified mixture is then filtered and, for example in a tubular reactor in a manner known per se, subjected to further transesterification until virtually complete conversion has been achieved. The reaction products can then, if desired, be subjected to fractional distillation or rectification and, if appropriate, be hydrogenated to the alcohols.

### EXAMPLES

### Example 1

### Fatty Acid Isolation ex Chaetoceros calcitrans/Simplex

Herbicide-resistant mutants of 2 microalgae from the aquaculture namely Chaetoceros calcitrans and Chaetoceros simplex (original strains available in the Northeast Pacific culture collection, British Columbia) were grown at 25° C. in artificial seawater (Harrison et al. J. Phycol. (1980) 16:28-35) in a 35 l tubular reactor (Bauart QVF) with an average light intensity of 100 μE/m² s. Beforehand, the scale-up of the inoculates took place at various stages analogously in stirred glass cells or fermenters in order to be able to operate the large reactor, start concentration of the algae mass 0.5 g/l. The pH in the system was kept constant at 8.2±0.2 by metering in air with 2% CO₂. At relative growth rates of >2 (doubling of the cell mass) and lipid contents of >50%, after 5 days the cell mass was harvested and processed. The lipids had the compositions (analyzed as total methyl ester following (trans)esterification with methanol) as in Table 1:

TABLE 1
Lipid compositions [% by wt.]

| Fatty acid methyl | | |
|---|---|---|
| ester content | Chaetoceros | Chaetoceros |
| (based on 100%) | calcitrans | simplex |
| Total C14 (sat. + | 28 | 35 |
| unsat.) | | |
| C16 | 54 | 53 |
| C18 | 3 | 4 |
| C20 | 12 | 6 |
| C22 | 1 | 1 |
| Uneven FA (Σ) | 2 | 2 |
| C15/C17 | | |

### Example 2

### Fatty Acid Isolation ex Isochyris sp.

A herbicide-resistant mutant of the microalgae Isochrysis sp. (T.ISO, CSRIO Algae Culture Collection) was grown over several stages to a feed material of 0.5 g/l reactor volume for a 30 l tubular reactor of the type (QVF). Here, an f2 culture medium (in accordance with Guillardt Ryther, Gran. Can. J. Microbiol. (1962) 18: 229-39) was used. The pH was adjusted to a pH of 8.5±0.2 through controlled introduction of a mixture of air with 5% CO₂. The fermentation takes place at a temperature of 30±2° C. The relative cell growth was 0.7 (doubling of the cell mass every three days), after 12 days, the biomass was harvested, the dry mass contained 35% lipid fraction, the fatty acid composition of the fatty acid constituents extracted and converted to methyl esters were—independently of the CO₂ concentration, at the values as in Table 2:

TABLE 2
Lipid compositions [% by wt.]

| Total contents of fatty | Isochrysis |
|---|---|
| acid methyl esters | sp. (T.510) |
| C14 | 29 |
| C16 | 41 |
| C18 | 19 |
| C20 | 1 |
| C22 | 9 |
| Other, uneven fatty acids | 1 |

### Example 3

### Influence of the Squalane Content on the Glycerol Deposition

The algae mass from example 1 with a natural squalane content of 0.4% by weight and an acid number of 4 was mechanically freed from water and transesterified in a twin-screw extruder with methanol and zinc acetate at a starting temperature of 180° C. Here, a degree of transesterification of 70% of theory was achieved. 30% by weight of methanol and also a 1% by weight zinc acetate, based on the feed stream of 5 kg/h, were metered in. After separating off the solids by filtration, a further reaction took place in a tubular reactor combined with in each case a separator for separating off the glycerol. A stirred reactor of 5 l was operated at 80° C. and a pressure of 2 bar with 1 kg of the solids-free product from the reaction in the extruder following (distillative) water removal with 30% strength by volume aqueous methanol and 0.3% strength by volume aqueous sodium methylate. After three hours, the glycerol phase settled out, and following the removal of methanol and washing with water and drying of the upper phase, 900 g of methyl ester with a residual content of bonded glycerol of 0.2% by weight were obtained, corresponding to a conversion of 99.8%.

### Comparative Example C1

An analogous lipid composition which was prepared by mixing corresponding plant triglycerides and was free from squalane was subjected to the same transesterification and processing as in example 3. The glycerol phase here settled out only after 3.5 h. 900 g of methyl ester with a residual content of bonded glycerol of 0.4% by weight were likewise obtained.

### Example 4

The algae mass from example 1 with a natural squalane content of 0.4% by weight and an acid number of 1.5 was mechanically freed from water and transesterified in a twin-screw extruder with methanol and sodium methylate at a starting temperature of 80° C. Here, a degree of transesterification of 70% of theory was achieved. 30% by weight of methanol and a 0.3% by weight of sodium methylate, based on the feed stream of 5 kg/h, were metered in. After separating off the solids by filtration, a further reaction took place. A stirred reactor of 5 l was operated at 80° C. and a pressure of 2 bar with 1 kg of the solids-free product from the reaction in the extruder following (distillative) water removal with 30% strength by volume aqueous methanol and 0.3% strength by volume aqueous sodium methylate. After three hours, the glycerol phase settled out, and after the removal of methanol and washing with water and drying of the upper phase, 900 g of methyl ester with a residual content of bonded glycerol of 0.2% by weight were obtained, corresponding to a yield of 99.8%.

### Comparative Example C2

An analogous lipid composition which was prepared by mixing corresponding plant triglycerides and was free from squalane was subjected to the same transesterification and processing as in example 3. The glycerol phase here settled out only after 3.5 h. 900 g of methyl ester with a residual content of bonded glycerol of 0.4% by weight were likewise obtained.

What is claimed is:

1-19. (canceled)

20. A lipophilic preparation consisting of:
(a) 20 to 40% by weight of myristic acid or esters thereof,
(b) 20 to 40% by weight of palmitic acid or esters thereof,
(c) 0.1 to 5% by weight of aliphatic and/or cycloaliphatic hydrocarbons, and
(d) less than 20% by weight of carboxylic acids or esters thereof having 12 or fewer carbons in the acyl moiety,
with the proviso that all of the percentages add up to 100% by weight.

21. The preparation of claim 20 wherein components (a), (b) and (d) are present independently of one another as full or partial glycerides.

22. The preparation of claim 20 wherein components (a), (b) and (d) are present independently of one another as esters with C1-C4 aliphatic alcohols.

23. The preparation of claim 20 wherein said hydrocarbon component (c) comprises squalenes and/or squalanes.

24. A process for producing a lipophilic preparation comprising the steps of:
(a) culturing lipid-producing single- or multi-celled microorganisms which have a lipid content of at least 10% by weight, based on dry mass, wherein the lipid fraction consists of:
(1) 20 to 40% by weight of myristic acid or esters thereof,
(2) 20 to 40% by weight of palmitic acid or esters thereof,
(3) 0.1 to 5% by weight of aliphatic and/or cycloaliphatic hydrocarbons, and
(4) less than 20% by weight of carboxylic acids or esters thereof having 12 or fewer carbons in the acyl moiety;
with the proviso that all of the percentages add up to 100% by weight; and
(b) extracting said microorganisms, wherein the lipid fraction is separated from the biomass.

25. The process of claim 24 wherein said microorganisms comprise microalgae.

26. The process of claim 25 wherein said microalgae are selected from the group consisting of Tetraselmis suecica, Nannochloropsis, Dinobryon divergens, Mallomonas caudata, Syncrypta globosa, Synura urella, Haptophycea isochrysis, Chaetos ceros, Paulova lutheri, Isochrysis galbana, Emiliana huxleyi, Prymnesiophycea parvum, and strains obtained therefrom by culturing or mutagenesis.

27. The process of claim 24 wherein said microorganisms are cultured in a photobioreactor.

28. The process of claim 24 wherein said microorganisms are cultured at a temperature in the range of from 20° to 40° C.

29. The process of claim 24 wherein said microorganisms are irradiated with daylight or an amount of artificial light from 200 to 1500 μm⁻² s⁻¹.

30. The process of claim 24 wherein said microorganisms are cultured in the presence of waste water from dairy or starch processing.

31. The process of claim 24 wherein said microorganisms are cultured in the presence of purified or unpurified carbon dioxide from combustion plants.

32. The process of claim 24 wherein said microorganisms are exposed to at least one stress factor during the growth phase.

33. The process of claim 32 wherein said at least one stress factor is selected from the group consisting of increasing light intensity, withdrawing nutrients, chemical stress, oxidative stress and changing the pH.

34. The process of claim 24 wherein, when the growth phase is complete, the suspension of the microorganisms is sedimented, filtered and/or centrifuged.
